(19) United States
(12) Patent Application Publication
(10) Pub. No.: US 2022/0117550 A1
(43) Pub. Date: Apr. 21, 2022

(54) Rehabilitation Support System and Rehabilitation Support Method

(71) Applicant: Nippon Telegraph and Telephone Corporation, Tokyo (JP)

(72) Inventors: Shin Toyota, Tokyo (JP); Takayuki Ogasawara, Tokyo (JP); Kenichi Matsunaga, Tokyo (JP)

(21) Appl. No.: 17/607,088

(22) PCT Filed: May 20, 2019
(86) PCT No.: PCT/JP2019/019887

§ 371 Date: Oct. 28, 2021

(51) Int. Cl. A61B 5/00; A61B 5/11; G16H 20/30

(52) U.S. Cl. CPC A61B 5/486; A61B 5/7275; A61B 2505/09; A61B 5/1116; G16H 20/30; A61B 5/742

(57) ABSTRACT

A rehabilitation support system includes: a sensor data acquirer that acquires sensor data that includes biometric information of a user measured by a sensor; a state calculator that obtains a state of the user based on the sensor data thus acquired; a predictor that predicts the state of the user based on the state of the user obtained by the state calculator; a storage unit that stores support information that is to be presented as information that supports rehabilitation; a selection unit that selects the support information stored in the storage unit, based on the state of the user predicted by the predictor; and a presentation unit that presents the support information selected by the selection unit.

### CROSS-REFERENCE TO RELATED APPLICATIONS

This application is a national phase entry of PCT Application No. PCT/JP2019/019887, filed on May 20, 2019, which application is hereby incorporated herein by reference.

### TECHNICAL FIELD

The present invention relates to a rehabilitation support system and a rehabilitation support method.

### BACKGROUND

With proper rehabilitation, patients and elderly people who need rehabilitation, for example, can recover physical functionality and achieve goals regarding the standard of living in mental and social aspects. Patients who need rehabilitation may need to engage in enthusiastic rehabilitation throughout their living hours to recover from illness, for example.

Conventionally, in the fields of sports and medical care, pieces of biometric information such as heart rate and the amount of activity measured by a sensor such as a wearable device have been utilized (see PTL 1 and NPL 1). For example, PTL 1 discloses a technology for more accurately analyzing the state of a patient's activity by focusing on lifestyle habits based on acceleration measured with sensors worn by the user.

According to conventional technology, it is possible to calculate the state of physical activity of a user such as a patient who carries out rehabilitation (hereinafter may simply be referred to as “rehab”) and present information such as the calculation result. However, active information the improves the user's motivation to actively engage in rehabilitation has not been provided to the user.

Therefore, for example, NPL 2 proposes a rehabilitation support technology for motivating the user to carry out rehabilitation when the user carries out rehabilitation training.

However, the conventional rehabilitation support technology provides support based on the user's rehabilitation records after the user has carried out rehabilitation training, and therefore, there is a problem in that the user cannot be provided with information that motivates the user to carry out rehabilitation before the user carries out rehabilitation.

### CITATION LIST

### Patent Literature

PTL 1 WO 2018/001740

### Non Patent Literature

NPL 1—KASAI, OGASAWARA, NAKASHIMA, TSUKADA, “Development of Functional Textile ‘hitoe’: Wearable Electrodes for Monitoring Human Vital Signals”, IEICE, Communications Society Magazine, NO. 41 (June 2017) (Vol. 11. No. 1)

NPL 2—SATO, OGASAWARA, TOYODA, MATSUNAGA, MUKAINO, “Feedback of activity monitoring results of rehabilitation patients”, 2019 IEICE General Conference (Information and Systems Lecture Proceedings 1) (D-7-5).

### SUMMARY

### Technical Problem

Embodiments of the present invention have been made to solve the above-described problem, and aims to provide a rehabilitation support technology that can more effectively motivate a user to engage in rehabilitation before the user carries out rehabilitation.

### Means for Solving the Problem

To solve the above-described problem, a rehabilitation support system according to embodiments of the present invention includes: a sensor data acquisition unit that acquires sensor data that includes biometric information of a user measured by a sensor; a state calculation unit that obtains a state of the user based on the sensor data thus acquired; a prediction unit that predicts the state of the user based on the state of the user obtained by the state calculation unit; a storage unit that stores support information that is to be presented as information that supports rehabilitation; a selection unit that selects the support information stored in the storage unit, based on the state of the user predicted by the prediction unit; and a presentation unit that presents the support information selected by the selection unit.

In the rehabilitation support system according to embodiments of the present invention, the state calculation unit may calculate a cumulative duration of the state of the user, and the prediction unit may predict a cumulative duration of the state of the user in a certain period in the future based on the cumulative duration of the state of the user calculated by the state calculation unit.

The rehabilitation support system according to embodiments of the present invention may further include a determination unit that determines whether or not the state of the user predicted by the prediction unit satisfies a condition that has been set regarding execution of rehabilitation, and the selection unit may select the support information corresponding to a result of the determination by the determination unit.

In the rehabilitation support system according to embodiments of the present invention, the determination unit may set a threshold value that is to be used for determination, based on information that includes statistical data regarding rehabilitation for each user.

In the rehabilitation support system according to embodiments of the present invention, the determination unit may set a threshold value that is to be used for determination, based on a history of the state of the user calculated by the state calculation unit.

In the rehabilitation support system according to embodiments of the present invention, the presentation unit may include a display device that displays an image that expresses the support information selected by the selection unit.

In the rehabilitation support system according to embodiments of the present invention, the state of the user may be at least one of a lying state, a standing state, a sitting state, and a walking state.

To solve the above-described problem, a rehabilitation support method according to embodiments of the present invention includes: a first step of acquiring sensor data that includes biometric information of a user measured by a sensor; a second step of obtaining a state of the user based on the sensor data thus acquired; a third step of predicting the state of the user based on the state of the user obtained in the second step; a fourth step of selecting support information that is stored in a storage unit and is to be presented as information that supports rehabilitation, based on the state of the user predicted in the third step; and a fifth step of presenting the support information selected in the fourth step.

### Effects of Embodiments of the Invention

According to embodiments of the present invention, the state of a user is estimated based on the state of user calculated based on sensor data that includes biometric information. Therefore, it is possible to more effectively motivate the user to engage in rehabilitation before the user carries out rehabilitation.

### BRIEF DESCRIPTION OF THE DRAWINGS

FIG. 1 is a block diagram showing a functional configuration of a rehabilitation support system according to a first embodiment of the present invention.

FIG. 2 is a block diagram showing an example of a computer configuration for realizing the rehabilitation support system according to the first embodiment.

FIG. 3 is a flowchart illustrating operations of the rehabilitation support system according to the first embodiment.

FIG. 4 is a diagram illustrating an outline of a specific example of a configuration of the rehabilitation support system according to the first embodiment.

FIG. 5 is a block diagram showing an example of a configuration of the rehabilitation support system according to the first embodiment.

FIG. 6 is a sequence diagram for the rehabilitation support system according to the first embodiment.

FIG. 7 is a block diagram showing a configuration of a rehabilitation support system according to a second embodiment.

FIG. 8 is a diagram illustrating a prediction unit according to the second embodiment.

FIG. 9 is a diagram illustrating the prediction unit according to the second embodiment.

FIG. 10 is a flowchart illustrating operations of the rehabilitation support system according to the second embodiment.

FIG. 11 is a block diagram showing a configuration of a rehabilitation support system according to a third embodiment.

FIG. 12 is a diagram illustrating a determination unit according to the third embodiment.

FIG. 13 is a diagram illustrating the determination unit according to the third embodiment.

FIG. 14 is a flowchart illustrating operations of the rehabilitation support system according to the third embodiment.

FIG. 15 is a flowchart illustrating operations of the rehabilitation support system according to the third embodiment.

FIG. 16 is a block diagram showing a configuration of a rehabilitation support system according to a fourth embodiment.

### DETAILED DESCRIPTION OF ILLUSTRATIVE EMBODIMENTS

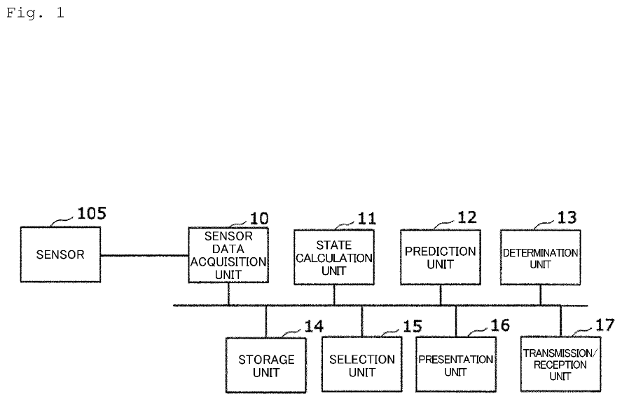

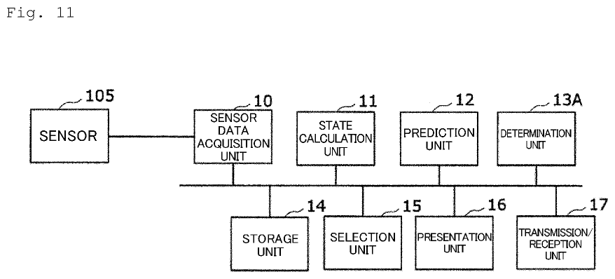

Hereinafter, preferred embodiments of the present invention will be described in detail with reference FIGS. 1 to 16. The following describes a case in which the user is a patient or the like who needs to carry out rehabilitation, and, for example, the patient carries out rehabilitation for getting up and walking because they spend a lot of time in a lying state.

### First Embodiment

First, an outline of a rehabilitation support system according to a first embodiment of the present invention will be described. FIG. 1 is a block diagram showing a functional configuration of the rehabilitation support system. The rehabilitation support system obtains the state of a user by acquiring sensor data measured by a sensor 105, which includes biometric information of the user. The rehabilitation support system also predicts the state of the user in a certain period of time in the future based on the history of the state of the user thus obtained. Furthermore, the rehabilitation support system determines whether or not the state of the user thus predicted satisfies a condition that has been set in advance regarding the rehabilitation carried out by the user, and selects and presents support information that is used to support rehabilitation, according to the result of the determination.

### Functional Blocks of Rehabilitation Support System

The rehabilitation support system includes a sensor data acquisition unit 10 that acquires data from the sensor 105, a state calculation unit 11, a prediction unit 12, a determination unit 13, a storage unit 14, a selection unit 15, a presentation unit 16, and a transmission/reception unit 17.

The sensor data acquisition unit 10 acquires sensor data that includes biometric information of the user, measured by the sensor 105. More specifically, if an acceleration sensor is attached to the user as the sensor 105, the sensor data acquisition unit 10 converts an analog acceleration signal measured by the acceleration sensor into a digital signal at a predetermined sampling rate. The biometric information measured by the sensor data acquisition unit 10 is to be stored in the storage unit 14 described below, in association with a measurement time.

In addition to the acceleration, the sensor data acquisition unit 10 may acquire an angular velocity, light, an electromagnetic wave, temperature and humidity, a pressure, positional information, a voice, a concentration, a voltage, a resistance, and the like as biometric information of the user. Also, the sensor data acquisition unit 10 can acquire electrocardiographic activity, myoelectric potential activity, a blood pressure, body gas exchanged through breathing, a body temperature, a pulse, and a brain wave as biometric information of the user, which can be obtained from the aforementioned physical quantities.

Also, in addition to the biometric information of the user, the sensor data acquisition unit 10 may acquire external environment data regarding the place where the user is present. External environment data includes, for example, information regarding the room temperature of the place where the user is present, the outside air temperature, the humidity, and the like. Note that the sensor data acquisition unit 10 may acquire pieces of biometric information of the user from a plurality of sensors 105 that each measure biometric information of the user, respectively.

The state calculation unit 11 calculates the state of the user based on the sensor data including the biometric information of the user, acquired by the sensor data acquisition unit 10. The state of the user refers to a posture, coordinates, a speed, a speech, breathing, walking, sitting, driving, sleeping, a body movement, a stress, and so on, which occur during rehabilitation carried out by the user and their daily life. The state of the user may also be the results of calculation performed on information or the like that indicates amounts such as the magnitude, frequency, increase and decrease, duration, and accumulation of the aforementioned factors.

Specifically, the state calculation unit 11 may estimate the state of the user by using, for example, the get-up state and the bed-rest state estimated using the acceleration of the user, disclosed in PTL 1. As a result of the state calculation unit 11 calculating the state of the user, it is possible to grasp the fact that the user is carrying out rehabilitation, the progress of the rehabilitation, and also the place where the user is present. Based on the state of the user thus calculated, it is possible to distinguish, for example, whether the user carried out rehabilitation in a training room with a doctor, or the user carried out rehabilitation in their own room or the like as voluntary training.

Also, the state calculation unit 11 may obtain the state of the user based on the biometric information of the user acquired over a period from the time when the sensor 105 has been attached to the user and the measurement has been started, to the latest measurement time. The state of the user calculated by the state calculation unit 11 is to be stored in the storage unit 14 together with time information.

The prediction unit 12 predicts the state of the user from the state of the user obtained by the state calculation unit 11. More specifically, the prediction unit 12 receives the state of the user calculated by the state calculation unit 11 as an input, and substitutes it into a preset model formula, and predicts and outputs the state of the user in a certain period of time in the future. For example, the prediction unit 12 can predicts the state of the user in the period of thirty minutes one or two hours after the current time.

In addition, the prediction unit 12 can use a model formula based on a prediction model constructed in advance through regression analysis or the like. For example, by using the state of the user in the past calculated by the state calculation unit 11 as an objective variable, it is possible to analyze the relationship between explanatory variables that are a plurality of factor candidates for the objective variable, calculate a coefficient for each factor candidate to calculate a regression formula, and use this formula as a model formula. Note that, in the present embodiment, the model formula has been stored in the storage unit 14 in advance.

Also, the model formula used by the prediction unit 12 may use, in addition to the history of the state of the user obtained by the state calculation unit 11, sensor data regarding the external environment such as the temperature measured by the sensor 105, the attributes of the user such as the age and the sex, and so on. For example, if the temperature of the place where the user is present is higher or lower than the range of 20° C. to 25° C. by at least certain degrees, for example, it is envisioned that the user is physically and psychologically uncomfortable when carrying out rehabilitation. In particular, when the user continuously carious out rehabilitation with a relatively low exercise load, such as sitting, standing, and walking, for a long period of time that is no shorter than a certain period of time, it is envisaged that the limit determined by psychological discomfort has a greater effect than the limit determined by physical factors.

Therefore, the prediction unit 12 can more precisely predict the state of the user in the future by using a model formula that takes the external environment such as the temperature and humidity, and the attributes of the user. In addition to the age and the sex, the height, the weight, illness details, the length of the hospital stay, and other medical examination information may be used as the attributes of the user. In addition, data that is based on these attributes of the user, such as the BMI, the degree of recovery, treatment history, and treatment transition, may also be used.

The determination unit 13 determines whether or not the state of the user predicted by the prediction unit 12 satisfies the condition that has been set regarding the execution of rehabilitation. The condition that has been set regarding the execution of rehabilitation is a condition that has been set to improve the user's motivation to carry out rehabilitation. The result of the determination by the determination unit 13 is input to the selection unit 15. In the present embodiment, the determination unit 13 reads out the condition stored in the storage unit 14 and performs determination processing.

Specifically, the determination unit 13 can determine whether or not the state of the user predicted by the prediction unit 12 is a specific state that has been set in advance. For example, a case where a user who spends a lot of time in a lying state carries out rehabilitation for getting up or walking is envisaged. In such a case, the determination unit 13 may determine whether or not the predicted state of the user is in a get-up state or a walking state that indicates the execution of rehabilitation.

The determination unit 13 can perform the determination by using not only the state of the user, but also a condition that uses time information, biometric information that is of a type different from the type of the specific biometric information used to calculate the state of the user, data regarding the external environment, and so on, as the condition set regarding the execution of rehabilitation. Also, the determination unit 13 may output the result of the determination based on a plurality of conditions.

The storage unit 14 stores the model formula for predicating the state of the user, which is to be used by the prediction unit 12. The storage unit 14 also stores the condition that has been set regarding the execution of rehabilitation and is to be used by the determination unit 13.

The storage unit 14 also stores rehabilitation support information. Rehabilitation support information is information that prompts the user to carry out rehabilitation. Rehabilitation support information may be a mode of an item that spatiotemporally changes, for example. An item is information that is in the form recognizable by a user, expressed in text, sound, vibration, heat, light, wind, stimulus, etc., and a combination thereof. For example, a specific scene from a moving image may be stored in the storage unit 14 as a mode of an item. In the present description, rehabilitation support information refers to a mode of an item such as an image.

The storage unit 14 also stores a mode of an item such as an image and the result of the determination in association with each other.

The storage unit 14 stores time-series data of the biometric information of the user acquired by the sensor data acquisition unit 10. The storage unit 14 also stores the history of the state of the user calculated by the state calculation unit 11. The calculated state of the user is stored in the storage unit 14 in association with the measurement time of the biometric information that is the basis therefor.

The selection unit 15 selects a mode of an item such as an image stored in the storage unit 14 according to the result of the determination by the determination unit 13. More specifically, if the predicted state of the user does not match a specific condition indicating the execution of rehabilitation, it is predicted that the user will not carry out rehabilitation in a certain period of time in the future. Therefore, the selection unit 15 selects a mode of an image that alerts the user, stored in the storage unit 14. If the result of the determination indicates that the user will be carrying out rehabilitation in the certain period of time in the future, the selection unit 15 can select a mode of an image stored in the storage unit 14, as rehabilitation support information that is to be presented in normal cases.

Also, the selection unit 15 can change the mode of the image by, for example, in a game-type application, providing a privilege or changing the content of the application.

The presentation unit 16 displays the mode of the item to be presented as rehabilitation support information selected by the selection unit 15 on a display device 109. For example, when getting up in the morning, the user sees the image or text information that is in a mode for prompting the rehabilitation, displayed on the display device 109, and thus the user is motivated to actively carry out rehabilitation for getting up, walking, and so on during the day.

The transmission/reception unit 17 receives sensor data that includes biometric information of the user, measured by the sensor 105. The transmission/reception unit 17 may convert the rehabilitation support information selected by the selection unit 15 according to predetermined communication standards, and transmit it to the presentation unit 16 connected to a communication network.

### Computer Configuration of Rehabilitation Support System

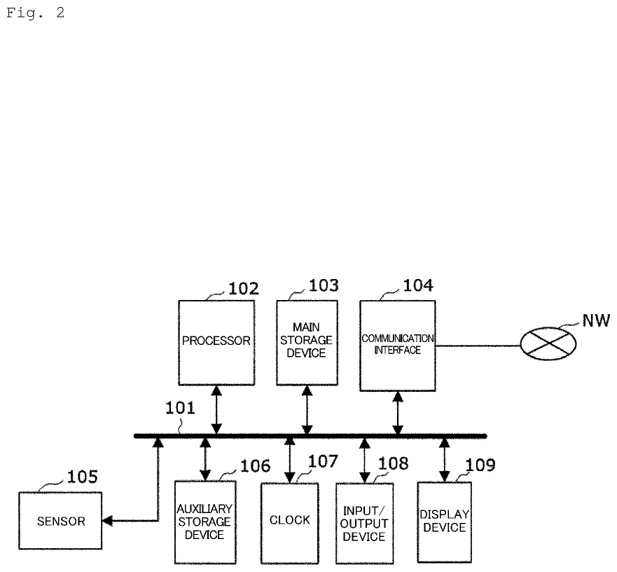

Next, a computer configuration for realizing the rehabilitation support system that has the above-described functions will be described with reference to FIG. 2.

As shown in FIG. 2, the rehabilitation support system can be realized using, for example, a computer that includes a processor 102, a main storage device 103, a communication interface 104, an auxiliary storage device 106, a clock 107, and an input/output device 108 that are connected with each other via a bus 101, and a program that controls these hardware resources. In the rehabilitation support system, for example, the sensor 105 provided outside the rehabilitation support system and the display device 109 that is provided inside the rehabilitation support system are connected with each other via the bus 101.

A program that is to be used by the processor 102 to perform various kinds of control and calculations has been stored in the main storage device 103 in advance. The processor 102 and the main storage device 103 realizes the functions of the rehabilitation support system including the state calculation unit 11, the prediction unit 12, the determination unit 13, and the selection unit 15 shown in FIG. 1.

The communication interface 104 is an interface circuit that is used to communicate with various external electronic devices via a communication network NW.

For example, an arithmetic interface and an antenna that conform to wireless data communication standards such as LTE, 3G, wireless LAN, or Bluetooth (registered trademark) may be used as the communication interface 104. The transmission/reception unit 17 illustrated in FIG. 1 is realized using the communication interface 104.

The sensor 105 is constituted by, for example, a heart rate monitor, an electrocardiograph, a sphygmomanometer, a pulse meter, a respiratory sensor, a thermometer, or an electroencephalogram sensor. More specifically, the sensor 105 is realized using a 3-axis acceleration sensor, a microwave sensor, a pressure sensor, an ammeter, a voltmeter, a thermo-hygrometer, a concentration sensor, a photo sensor, or a combination thereof.

The auxiliary storage device 106 is constituted by a readable and writable storage medium and a drive device for reading and writing various kinds of information such as programs and data from and to the storage medium. A hard disk or a semiconductor memory such as a flash memory may be used as the storage medium in the auxiliary storage device 106.

The auxiliary storage device 106 has a storage area for storing the biometric information measured by the sensor 105, and a program storage area for storing a program to be used by the rehabilitation support system to perform processing to analyze the biometric information. The storage unit 14 illustrated in FIG. 1 is realized using the auxiliary storage device 106. Furthermore, the auxiliary storage device 106 may also have a backup area for backing up the above-described data and program.

The clock 107 is constituted by, for example, a built-in clock that is built into the computer, and measures the time. Alternatively, the clock 107 may acquire time information from a time server (not shown). Time information acquired by the clock 107 is recorded in association with the calculated state of the user. Time information acquired by the clock 107 is also used to sample biometric information.

The input/output device 108 is constituted by an I/O terminal that inputs a signal from an external device such as the sensor 105 or the display device 109 and outputs a signal to an external device.

The display device 109 is realized using a liquid crystal display or the like. The display device 109 realizes the presentation unit 16 illustrated in FIG. 1.

### Rehabilitation Support Method

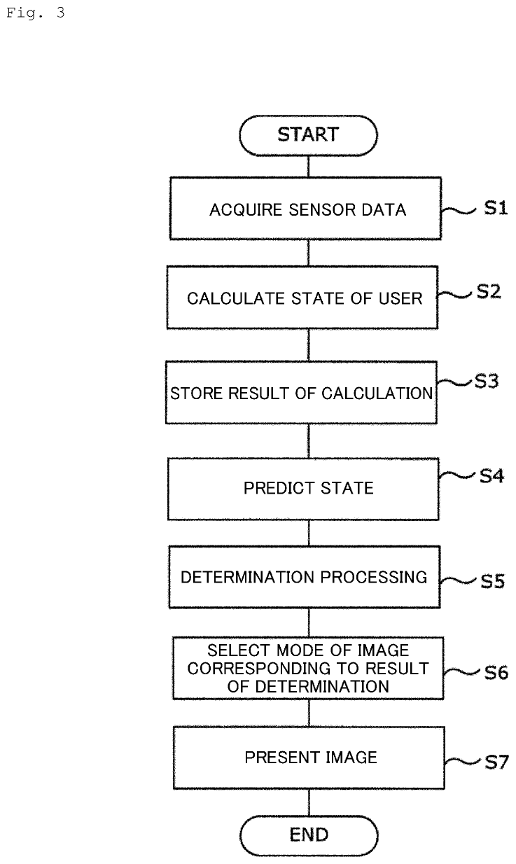

Next, operations of the rehabilitation support system with the above-described configuration will be described with reference to the flowchart shown in FIG. 3. First, the following processing is performed in a state where the sensor 105 is attached to the user, for example.

The sensor data acquisition unit 10 acquires the biometric information of the user measured by the sensor 105, via the transmission/reception unit 17 (step S1). The acquired biometric information is to be accumulated in the storage unit 14. Note that the sensor data acquisition unit 10 can remove noise from the acquired biometric information, and perform processing to convert the biometric information in the form of an analog signal into digital signal.

Next, the state calculation unit 11 calculates the state of the user based on the biometric information of the user, acquired by the sensor data acquisition unit 10 (step S2). For example, the state calculation unit 11 calculates the state of the user, which is a lying state, a get-up state, or a walking state, from the data indicating the acceleration of the user, acquired by the sensor data acquisition unit 10. The result of the calculation by the state calculation unit 11 is to be stored in the storage unit 14 together with time information (step S3).

Thereafter, the prediction unit 12 reads out the model formula for predicting the state of the user from the storage unit 14 to predict the state of the user (step S4). More specifically, the prediction unit 12 performs computation according to the model formula, using the history of the state of the user calculated in step S2, as an input, and outputs a prediction value regarding the state of the user in a certain period of time in the future. The predicted state of the user is to be stored in the storage unit 14. The prediction value regarding the state of the user is a predication value regarding a given period of time in the future such as a period of thirty minutes after one or two hours from the current time.

Next, the determination unit 13 reads out the condition set regarding the execution of rehabilitation from the storage unit 14, and determines whether or not the state of the user predicted in step S4 satisfies the set condition (step S5). For example, in a case where a user who spends a lot of time in a lying state carries out rehabilitation for getting up, if the predicted state of the user in a certain period of time in the future is a lying state, the determination unit 13 can output the result of the determination indicating that the user will not carry out rehabilitation in the future.

Thereafter, the selection unit 15 selects a mode of an item that is to be presented as rehabilitation support information, according to the result of the determination (step S6). More specifically, the selection unit 15 selects a mode of an image stored in the storage unit 14 in association with the result of the determination. For example, a case of the result of the determination indicating that the user will not carry out rehabilitation in the period of thirty minutes after two hours from the current time is envisaged. If this is the case, the selection unit 15 can selects an image, text, or audio that is in a mode that prompts the user to carry out rehabilitation or alerts the user, as well as vibration, heat, light, or the like added thereto. The image may be a moving image, a still image, or a stereoscopic image.

Next, the presentation unit 16 displays the mode of an item or the like selected by the selection unit 15, on the display device 109 (step S7). For example, the display device 109 displays a moving image scene of an animation character walking, as well as text information “Let's go for a walk today”.

Note that such an item like an image may be displayed in addition to a mode of an image that is normally displayed in a rehabilitation support application or the like. For example, in a rehabilitation support application, when a game format or a predetermined story is used, an item may indicate that points will be given, or may be switched to a special image. In addition, it is possible to present rehabilitation support information using an item in a form that can be recognized by the user.

### Specific Configuration of Rehabilitation Support System

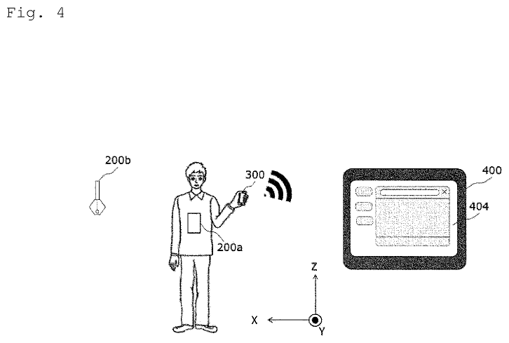

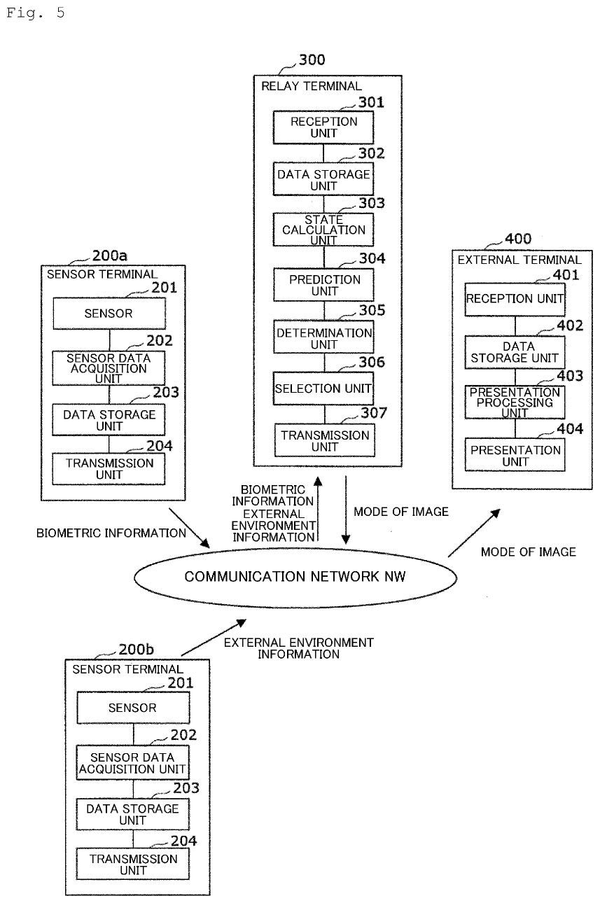

Next, a specific example of the above-described configuration of the rehabilitation support system will be described with reference to FIGS. 4 and 5.

For example, as shown in FIG. 4, the rehabilitation support system includes a sensor terminal 200a that is to be attached to a user who carries out rehabilitation, a sensor terminal 200b that measures external environmental data regarding the place where the user is present, a relay terminal 300, and an external terminal 400. One or more or all of the sensor terminals 200a and 200b, the relay terminal 300, and the external terminal 400 have the functions included in the rehabilitation support system such as the state calculation unit 11, the prediction unit 12, the determination unit 13, and the selection unit 15 illustrated in FIG. 1. Hereinafter, the relay terminal 300 includes the state calculation unit 11, the prediction unit 12, the determination unit 13, and the selection unit 15 illustrated in FIG. 1, and rehabilitation support information is presented to the external terminal 400.

### Functional Blocks of Sensor Terminals

As shown in FIG. 5, the sensor terminals 200a and 200b each include a sensor 201, a sensor data acquisition unit 202, a data storage unit 203, and a transmission unit 204. The sensor terminal 200a is placed on the trunk of the user's body, and measures biometric information such as an acceleration and a body temperature, for example. The sensor terminal 200b measures external environment data, such as the humidity and the temperature in the place where the user is present. The sensor terminals 200a and 200b transmits the measure biometric information of the user and external environment data to the relay terminal 300 via the communication network NW.

Each sensor 201 is realized using a 3-axis acceleration sensor, for example. As shown in FIG. 5, for example, the three axes of the acceleration sensor included in each sensor 201 are provided such that the X-axis is parallel with the left-right direction of the body, the Y-axis is parallel with the front-rear direction of the body, and the Z-axis is parallel with the top-bottom direction of the body. Each sensor 201 corresponds to the sensor 105 illustrated in FIGS. 1 and 2.

Each sensor data acquisition unit 202 acquires the biometric information and external environment data measured by the sensors 201. Each sensor data acquisition unit 202 performs noise removal and sampling processing on the acquired biometric information, and obtains time series data in the biometric information in the form of a digital signal, for example. Each sensor data acquisition unit 202 corresponds to the sensor data acquisition unit 10 illustrated in FIG. 1.

Each data storage unit 203 stores time-series data such as the biometric information and external environment data measured by the sensor 201, the biometric information in the form of a digital signal pressed and acquired by the sensor data acquisition unit 202. Each data storage unit 203 corresponds to the storage unit 14 (FIG. 1).

Each transmission unit 204 transmits the biometric information and external environment data stored in the data storage unit 203 to the relay terminal 300 via the communication network NW. Each transmission unit 204 includes, for example, a communication circuit for performing wireless communication conforming to wireless data communication standards such as LTE, 3G, wireless LAN (Local Area Network) or Bluetooth (registered trademark). Each transmission unit 204 corresponds to the transmission/reception unit 17 (FIG. 1).

### Functional Blocks of Relay Terminal

The relay terminal 300 includes a reception unit 301, a data storage unit 302, a state calculation unit 303, a prediction unit 304, a determination unit 305, a selection unit 306, and a transmission unit 307. The relay terminal 300 analyzes the biometric information of the user received from the sensor terminal 200a. The relay terminal 300 also calculates the state of the user based on the biometric information of the user. The relay terminal 300 also predicts the state of the user in a certain period of time in the future based on the state of the user thus calculated, and determines whether or not the predicted state of the user satisfies a condition that has been set in advance. The relay terminal 300 also selects a mode of an image according to the result of the determination. Information indicating the selected mode of an image is transmitted to the external terminal 400.

The relay terminal 300 is realized using a smartphone, a tablet, a laptop personal computer, a gateway, or the like.

The reception unit 301 receives biometric information and external environment data from the sensor terminals 200a and 200b via the communication network NW. The reception unit 301 corresponds to the transmission/reception unit 17 (FIG. 1).

The data storage unit 302 stores the biometric information of the user and the external environment data received by the reception unit 301 and the history of the state of the user in a measurement period estimated by the data analyzing unit 303. The data storage unit 302 corresponds to the storage unit 14 (FIG. 1).

The state calculation unit 303, the prediction unit 304, the determination unit 305, and the selection unit 306 respectively correspond to the functional units illustrated in FIG. 1.

The transmission unit 307 transmits information indicating a mode of an item such as an image to be presented as rehabilitation support information, selected by the selection unit 306, to the external terminal 400 via the communication network NW. The transmission unit 304 corresponds to the transmission/reception unit 17 (FIG. 1).

### Functional Blocks of External Terminal

The external terminal 400 includes a reception unit 401, a data storage unit 402, a presentation processing unit 403, and a presentation unit 404. The external terminal 400 generates and presents rehabilitation support information based on information received from the relay terminal 300 via the communication network NW.

As with the relay terminal 300, the external terminal 400 is realized using a smartphone, a tablet, a laptop personal computer, a gateway, or the like. The external terminal 400 is provided with the display device 109, which generates and displays a mode of an item that has been received, such as an image. The display device 109 may present the selected mode of an item as rehabilitation support information, using an audio output device, a light source or the like, instead of the display device 109.

The reception unit 401 receives information indicating the mode of an item such as an image to be presented as rehabilitation support information, from the relay terminal 300 via the communication network NW. The reception unit 401 corresponds to the transmission/reception unit 17 (FIG. 1).

The data storage unit 402 stores a mode of an item such as an image. The data storage unit 402 corresponds to the storage unit 14 (FIG. 1).

The presentation processing unit 403 reads out a mode of an item such as an image to be presented as rehabilitation support information, from the data storage unit 402, and outputs it. The presentation processing unit 403 can generate a mode of an image corresponding to the result of the determination by the determination unit 305 (the determination unit 13), and control the display format of rehabilitation support information. The presentation processing unit 403 may also read preset materials such as images, moving images, audio, or the like, synthesize a moving image and audio to be presented, set the playback speed, process it using an effect filter or the like, and encode the edited results. The presentation processing unit 403 is a function included in the presentation unit 16 illustrated in FIG. 1.

The presentation unit 404 outputs an item such as an image in the selected mode as rehabilitation support information according to an instruction from the presentation processing unit 403. The presentation unit 404 may display a scene from a moving image and text information prompting the user to carry out rehabilitation, on the display device 109, or may output audio from a speaker (not shown) provided in the external terminal 400. In addition, the presentation unit 404 may present rehabilitation support information by using a method employing vibration, light, stimulation, or the like that is recognizable by the user. The presentation unit 404 may also display information indicating the external environment such as the temperature measured by the sensor terminal 200b, together with an image showing a selected scene from a moving image. The presentation unit 404 corresponds to the presentation unit 16 illustrated in FIG. 1.

As described above, the rehabilitation support system according to embodiments of the present invention has a configuration in which the functions shown in FIG. 1 are dispersed as the sensor terminals 200a and 200b, the relay terminal 300, and the external terminal 400. The rehabilitation support system according to embodiments of the present invention dispersedly performs processing related to the acquisition of biometric information of a user, the calculation of the state of the user, the prediction of the state of the user in a certain period of time in the future, determination processing, and furthermore the selection of a mode of an item such as an image corresponding to the result of the determination, the generation of an image in the selected mode, and the presentation thereof.

### Operation Sequence of Rehabilitation Support System

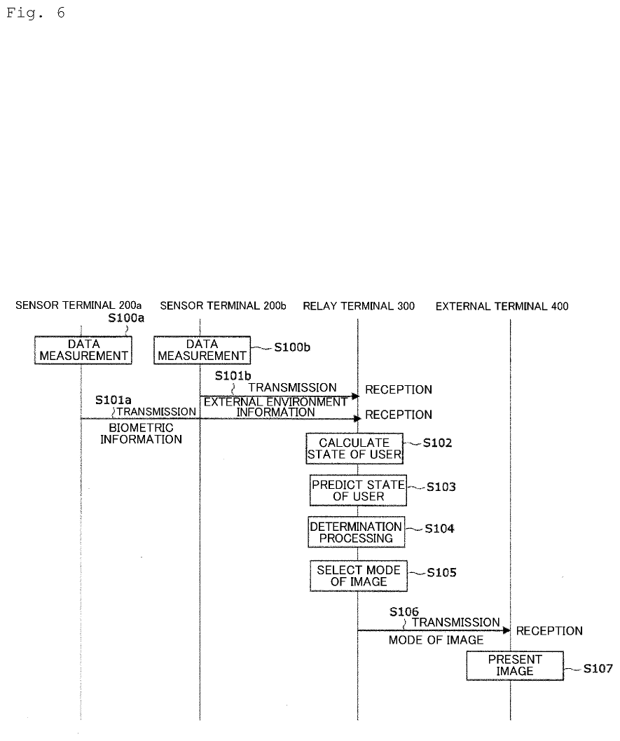

Next, operations of the rehabilitation support system with the above-described configuration will be described with reference to the sequence diagram shown in FIG. 6.

As shown in FIG. 6, first, the sensor terminal 200a is attached to the user, and measures biometric information such as a 3-axis acceleration (step S100a). The sensor terminal 200a obtains a digital signal of the measured biometric information, and performs noise removable when necessary. Next, the sensor terminal 200a transmits the biometric information to the relay terminal 300 via the communication network NW (step S101a).

Meanwhile, the sensor terminal 200b is installed in the place where the user is present, and measures data indicating the external environment such as the temperature (step S100b). The information indicating the measured external environment is transmitted to the relay terminal 300 via the communication network NW (step S101b).

Thereafter, the relay terminal 300, upon receiving the biometric information from the sensor terminal 200a, calculates the state of the user based on the biometric information (step S102). More specifically, the data analyzing unit 303 of the relay terminal 300 calculates the state of the user resulting from rehabilitation or daily life from the biometric information and the external environment data, and records it together with time information regarding the time at which the biometric information that is the basis for the state of the user was measured.

Next, the prediction unit 304 carries out the model formula set in advance, using the state of the user calculated in step S102 as an input, to predict the state of the user in a certain period of time in the future (step S103). Note that the model formula is stored in the data storage unit 302. The prediction unit 304 may predict the state of the user by using a model formula that also uses external environment data as an input.

Thereafter, the determination unit 305 determines whether or not the predicted state of the user satisfies the condition that has been set (step S104). Next, the selection unit 306 selects a mode of an item such as an image corresponding to the result of the determination (step S105).

Thereafter, the relay terminal 300 transmits information indicating the selected mode of an item to the external terminal 400 via the communication network NW (step S106). At this time, information indicating the external environment measured by the sensor terminal 200b may also be transmitted to the external terminal 400. The external terminal 400, upon receiving the information indicating the mode of an item, performs processing to present the item to be presented as rehabilitation support information (step S107).

As described above, the rehabilitation support system according to the first embodiment calculates the state of the user resulting from rehabilitation and daily life based on the biometric information of the user measured by the sensor 105, and predicates the state of the user in a certain period of time in the future based on the calculated state of the user. The rehabilitation support system also determines whether or not the result of the prediction satisfies the condition that has been set regarding the execution of rehabilitation, and selects and present a mode of an item such as an image according to the result of the determination. Therefore, it is possible to perform rehabilitation support to motivate the user to engage in rehabilitation before the user carries out rehabilitation.

### Second Embodiment

Next, a second embodiment of the present invention will be described. Note that, in the following description, the same components as in the above-described first embodiment are given the same reference signs and descriptions thereof are omitted.

In the second embodiment, a prediction unit 12A calculates a prediction value of a cumulative duration of the state of a user in a certain period of time in the future, using a model formula that employs a cumulative duration of the state of the user calculated by the state calculation unit 11, as input data.

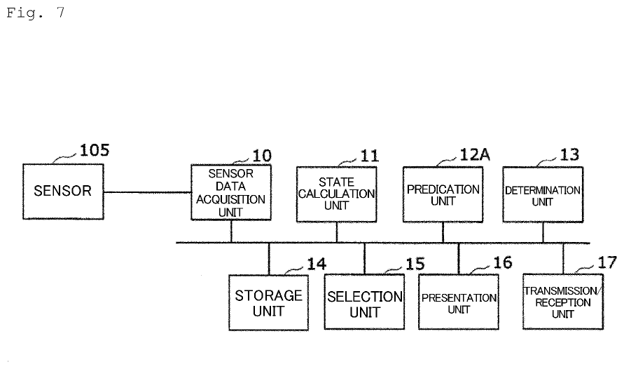

As shown in FIG. 7, the state calculation unit 11 calculates the state of the user based on the biometric information of the user acquired by the sensor data acquisition unit 10, and also calculates the cumulative duration of the state. For example, the state calculation unit 11 can calculate that the user is in a lying state, a standing state, a sitting state, or a walking state based on acceleration data, and obtain the cumulative duration of each state.

For example, the state calculation unit 11 may calculate the cumulative duration of each of the lying state, standing state, sitting state, and walking state of the user in the most recent day. Specifically, it is possible to obtain the most recent duration of sleep of the user by calculating the cumulative duration of the lying state of the user (for example, the time slot from 21:30 on the previous day to 8:00 on the current day).

The prediction unit 12A predicts the cumulative duration of the state of the user in a certain period of time in the future based on the cumulative duration of the state of the user calculated by the state calculation unit 11. Specifically, the prediction unit 12A obtains the prediction value of the cumulative duration of the state of the user in a certain period of time in the future by using a model formula that is based on the relationship between the cumulative duration of the state of the user calculated by the state calculation unit 11 and the cumulative duration of the state of the user in a certain period of time.

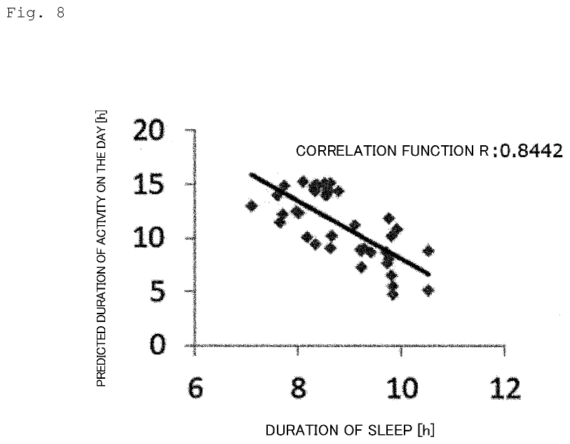

For example, as shown in FIG. 8, it is possible to perform simple regression analysis, where the duration of sleep (h) is expressed as an explanatory variable x and the duration of activity (h) of the day is expressed as an objective variable y, based on a graph plotting the relationship between the duration of sleep on the most recent day (the previous day) and the duration of activity on the day, of forty hospitalized patients, and use the regression line thus obtained (y=−1.324x+24.3465) as a model formula. Here, the duration of activity means the duration of a state in which the user is performing a get-up or walking action, i.e., the duration of a state in which the user is carrying out rehabilitation.

According to the example of a model formula shown in FIG. 8, the error between the predicted value of the duration of activity of the user on the day and the actually measured duration of activity of the user is 13%. Here, the error is defined as the mean of (|residual|/actual data value)×100.

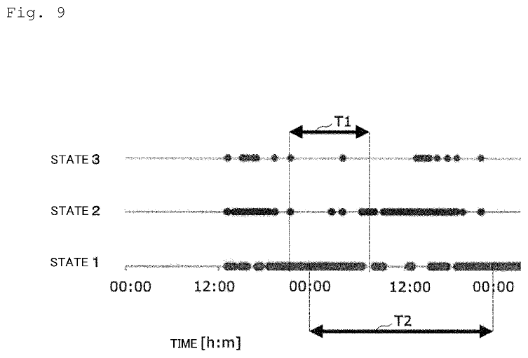

As shown in FIG. 9, the duration of sleep of the user on the previous day and the predicted duration of activity on the day shown in FIG. 8 are obtained from the total duration of each state of the user in a preset time slot. In FIG. 9, a state 1 shows a state in which the user is lying down, such as a lying state, a state 2 shows a state in which the user has got up, and a state 3 indicates a state in which the user is walking. The horizontal axis indicates time (hours : minutes) and indicates points in time at which the states occurred in two days.

For example, the case of predicting the duration of activity of a user on a certain day is envisaged. Here, the duration of activity is the cumulative duration of the state in which the user has got up (the state 2) and the state in which the user is walking (the state 1).

The total time of the duration of sleep of the user is calculated as the total duration of the state in which the user was lying down (the state 1) during a time slot T1 (from 21:30 on the previous day (the first day) to 8:00 on the day (the second day)). Also, the total duration of activity of the user is calculated as the cumulative duration of the get-up state (the state 2) and the walking state (the state 1) in twenty-four hours (a time slot T2) on the day (the second day).

Note that the number of variables and units used in the model formula can be freely determined. In the example shown in FIG. 8, regarding the state of the user in the future, the duration of activity of the user in the 24-hour time slot from twelve midnight of the day is predicted. However, any time unit such as day, day of the week, week, month, or year can be used to define the time slot.

The cumulative duration of each state of the user shown in FIG. 9 is calculated by the state calculation unit 11 and is stored in the storage unit 14. The model formula used by the prediction unit 12A is also stored in the storage unit 14 in advance.

The state of the user predicted by the prediction unit 12A and the state of the user given to the model formula as an input may be the same or different as long as they are correlative. For example, in the example in FIG. 9, the relationship between the lying state (the duration of sleep) and the get-up and walking states (the duration of activity) of the user are used. However, for example, it is possible to predict the duration of waking in the future by using the duration of walking in the past as an input.

### Rehabilitation Support Method

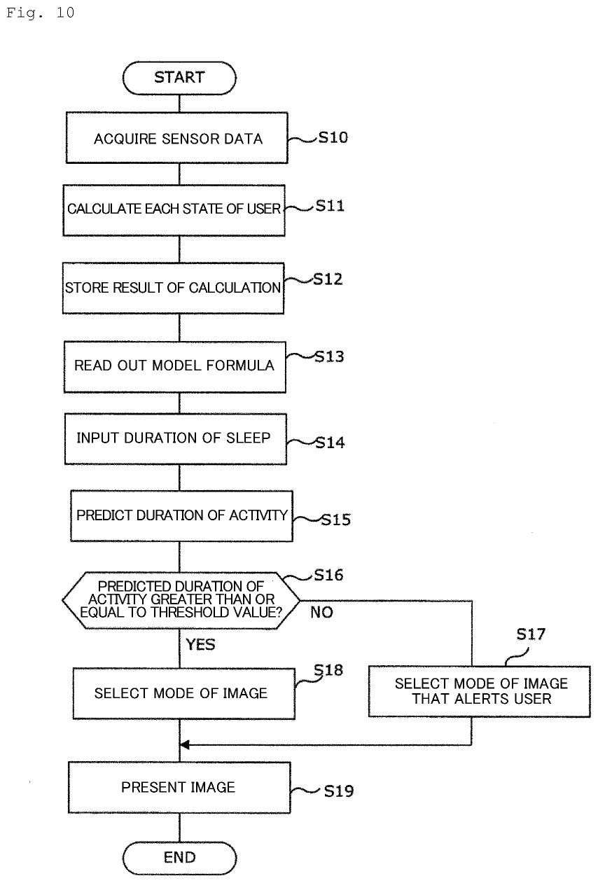

Next, operations of the rehabilitation support system with the above-described configuration will be described with reference to the flowchart shown in FIG. 10. As a premise, it is assumed that the storage unit 14 stores a model formula for prediction that is to be used by the prediction unit 12A. The storage unit 14 also stores a threshold value that is to be used as a condition that has been set regarding the execution of rehabilitation and is to be used by the determination unit 13. First, the following processing is performed in a state where the sensor 105 is attached to the user, for example.

The sensor data acquisition unit 10 acquires the biometric information of the user measured by the sensor 105, via the transmission/reception unit 17 (step S10). The acquired biometric information is to be accumulated in the storage unit 14.

Next, the state calculation unit 11 calculates the state of the user based on the biometric information of the user, acquired by the sensor data acquisition unit 10 (step S11). For example, the state calculation unit 11 calculates the state of the user, such as a lying state, a get-up state, or a walking state, or a walking state from the data indicating the acceleration of the user, acquired by the sensor data acquisition unit 10. The result of the calculation by the state calculation unit 11 is to be stored in the storage unit 14 together with time information (step S12). The storage unit 14 stores the cumulative duration of each state of the user.

Thereafter, the prediction unit 12A reads out the model formula for predicting the state of the user from the storage unit 14 to predict the state of the user (step S13). Specifically, the prediction unit 12A uses the model formula that utilizes the relationship between the duration of sleep of the user on the previous day and the predicted duration of activity on the day, which is shown in FIG. 8 and is stored in the storage unit 14. The prediction unit 12A reads out the duration of sleep (the cumulative duration of the lying state) of the user from the previous day to the day from the storage unit 14, and input it to the model formula (step S14). Specifically, the prediction unit 12A inputs the cumulative duration of the lying state of the user in the time slot from 21:30 on the previous day to 8:00 on the day, to the model formula.

Thereafter, the prediction unit 12A outputs the predicted duration of activity of the user on the day (step S15). The predicted duration of activity of the user is to be stored in the storage unit 14.

Next, if the determination unit 13 determines that the duration of activity of the user on the day predicted in step S15 is lower than a threshold value that has been set regarding the execution of rehabilitation (step S16: NO), the selection unit 15 selects a mode of an item such as an image that prompts the user to carry out rehabilitation, from among items such as images stored in the storage unit 14 (step S17). For example, in the case of the user carrying out rehabilitation for getting up or walking, if the predicted duration of activity of the user on the day is lower than the threshold value, it can be predicted that the user will not carry out sufficient rehabilitation on the day. Therefore, the selection unit 15 selects a mode such as a specific image or text for prompting the user to carry out rehabilitation or alert the user.

On the other hand, if the duration of activity of the user on the day predicted by the prediction unit 12A is higher than or equal to the threshold value (step S16: YES), the selection unit 15 selects a mode of an item such as an image that is to be present as rehabilitation support information in normal cases (step S18). Thereafter, the presentation unit 16 displays the mode of an item or the like selected by the selection unit 15, on the display device 109 (step S19).

As described above, with the rehabilitation support system according to the second embodiment, the cumulative duration of a predetermined state in a certain period of time in the future is predicted based on the cumulative duration of the state of the user calculated by the state calculation unit 11. Therefore, it is possible to grasp the duration of activity or the like of the user in any time slot in the future, such as in a day, a day of the week, a week, or a month, and it is possible to provide more effective preparatory rehabilitation support.

### Third Embodiment

Next, a third embodiment of the present invention will be described. Note that, in the following description, the same components as in the above-described first and second embodiments are given the same reference signs and descriptions thereof are omitted.

A determination unit 13A in the third embodiment is different from the determination unit 13 in the first and second embodiments in that the determination unit 13A performs determination processing using a condition regarding the execution of rehabilitation that has been set for each user.

The determination unit 13A sets a threshold value to be used for determination, based on information that includes statistical data regarding rehabilitation for each user. The determination unit 13A reads out the threshold value set for each user regarding the execution of rehabilitation, from the storage unit 14, and performs threshold value processing regarding the state of the user in a certain period in the future predicted by the prediction unit 12.

It is preferable that an appropriate target value is set for rehabilitation according to attributes of the user such as the degree of recovery and symptoms. For example, a threshold value that is based on the value of FIM (Functional Independence Measure) can be set. FIM is an indicator used to evaluate how much a patient (user) who is carrying out rehabilitation can perform activities in daily life by themselves. In the following example, the degree of recovery of the user who carries out rehabilitation is used as an indicator for evaluation. However, another indicator for evaluation such as a heart rate, a body movement, an elapsed time, a stress value, or screening may also be used in addition to the FIM value.

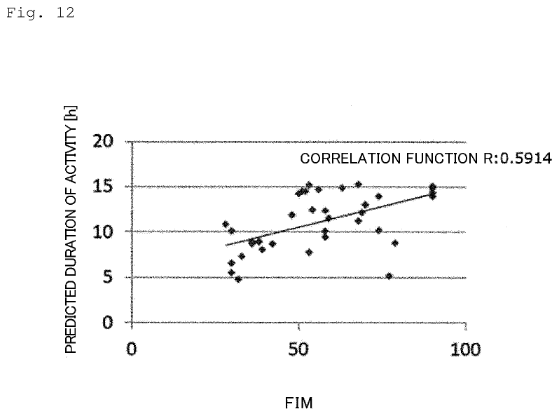

FIG. 12 shows a relationship between an FIM value and the duration of activity of the user who carries out rehabilitation on the day. A regression equation of the FIM value (x) and the duration of activity (y) of the user is derived from the relationship shown in the example in FIG. 12, and a model formula (y=0.094x+5.865) for setting a threshold value for each user is set based on this regression equation. For example, the predicted duration of activity of a user whose FIM value is 51 is calculated as 10.659 (h). The determination unit 13A can set this predicted duration as a threshold value to perform determination processing.

For example, in the example of the model formula for setting the threshold value shown in FIG. 12, if the duration of activity of the user predicted by the prediction unit 12 is longer than the predicted duration of activity (threshold value) obtained from the FIM value of the user, the determination unit 13A can output a result of the determination indicating “OK”.

As shown in FIG. 12, when the threshold value to be used by the determination unit 13A is set for each user, the proportion of the users for which the result of the determination is “No” even though the actual duration of activity of the user is greater than or equal to the threshold value, or for which the threshold value is “Yes” even though the duration of activity is less than the threshold value, to samples, was 28%.

Note that the specific value of the threshold value set for the execution of rehabilitation can be freely set according to the target value of rehabilitation that differs for each user. For example, the threshold value can be adjusted according to how long the user is desired to perform activity such as getting up or walking relative to the predicted duration of activity (threshold value) obtained from the FIM value of the user. It is also possible to adjust the threshold value and determine the minimum duration of daily rehabilitation based on the threshold value. Thus, the threshold value can be further adjusted by using a correction value α (h) set for each user. In such a case, the determination unit 13A executes processing to determine the predicted state of the user by using a value obtained by adding the correction value α to the threshold value.

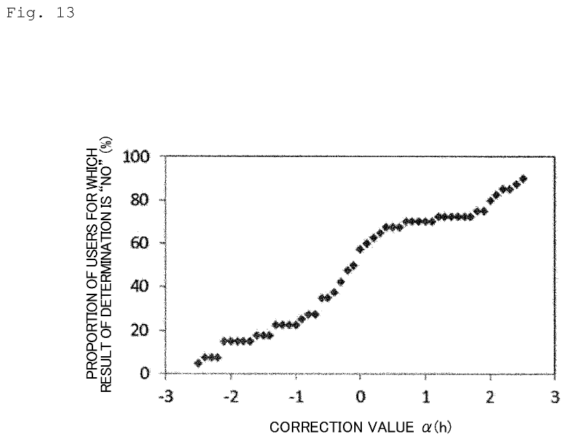

FIG. 13 shows the correction value α and the proportion of the users for which the result of the determination is “NO” to the total number of samples. In the example in FIG. 13, the proportion of the users for which the result of the determination is “No” increases as the correction value α increases. That is to say, the number of users to which a mode of an item such as an image that prompts the users to carry out rehabilitation is presented increases as the correction value α increases.

For example, the case of predicting, from the duration of sleep of a user on the previous day, the duration of activity in twenty-four hours on the next day according to the example in FIG. 8 is envisaged. For example, a mode of an item such as an image that prompts a user to carry out rehabilitation is presented to users whose duration of activity predicted by the prediction unit 12 is shorter than the predicted duration of activity (threshold value) obtained from the FIM value by one hour or more. In this case, the determination unit 13A sets −1 to the correction value α. As a result of adjusting the threshold value using the correction value α, as shown in FIG. 13, the result of the determination is “NO” for 20% of the total number of sample users. Therefore, a mode of an item such as an image that prompts a user to carry out rehabilitation is presented to 20% of the total number of users.

Note that the correction value α can be freely set according to the duration of activity that is expected for a user, the proportion of the number of users to which a mode of an item such as an image that encourages or prompts users to carry out rehabilitation is to be presented, or the like.

### Rehabilitation Support Method

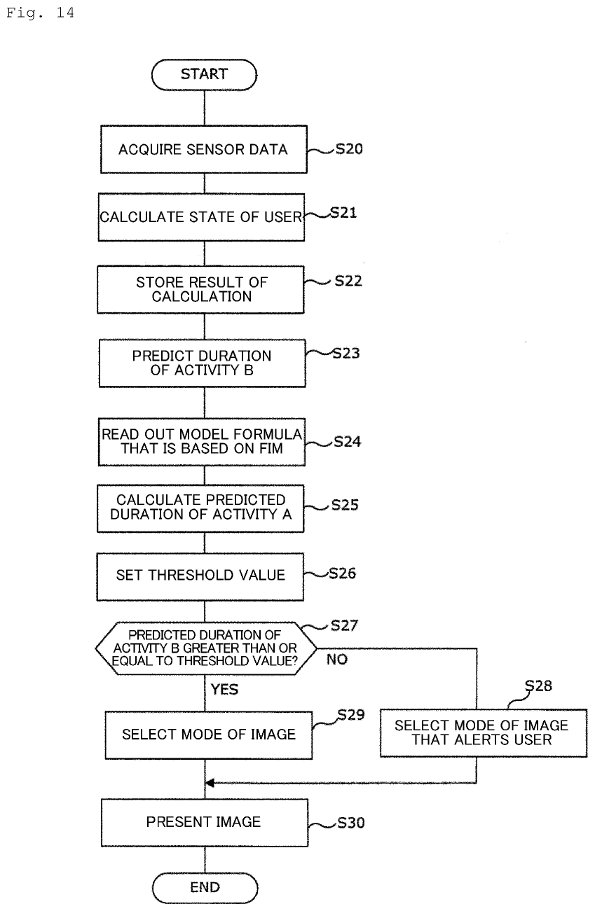

Next, specific operations of the rehabilitation support system with the above-described configuration will be described with reference to the flowchart shown in FIG. 14. Hereinafter, as a premise, the storage unit 14 stores a threshold value that is to be used by the determination unit 13A and is based on the FIM value of a user that has been set regarding the execution of rehabilitation. The storage unit 14 also stores a model formula that is to be used by the prediction unit 12 to predict the state of the user. First, the following processing is performed in a state where the sensor 105 is attached to the user, for example.

The sensor data acquisition unit 10 acquires the biometric information of the user measured by the sensor 105, via the transmission/reception unit 17 (step S20). The acquired biometric information is to be accumulated in the storage unit 14.

Next, the state calculation unit 11 calculates the state of the user based on the biometric information of the user, acquired by the sensor data acquisition unit 10 (step S21). For example, the state calculation unit 11 calculates the state of the user, such as a lying state, a get-up state, or a walking state, and a walking state from the data indicating the acceleration of the user, acquired by the sensor data acquisition unit 10. The result of the calculation by the state calculation unit 11 is to be stored in the storage unit 14 together with time information (step S22). The storage unit 14 stores the cumulative duration of each state of the user.

Thereafter, the prediction unit 12 reads out the model formula to be used to predict the state of the user from the storage unit 14, and outputs the predicted duration of activity B of the user on the day by using, as an input, the cumulative duration of the lying state of the user calculated by the state calculation unit 11 (step S23). The duration of activity B is to be stored in the storage unit 14.

Thereafter, the determination unit 13A reads out the model formula for setting the threshold value that is based on the FIM value of the user, from the storage unit 14 (step S24). Next, the determination unit 13A calculates a predicted duration of activity A according to the FIM value of the user shown in the example in FIG. 12 (step S25). Next, the determination unit 13A sets a threshold value that is based on the predicted duration of activity A (step S26). Thereafter, if the determination unit 13A determines that the duration of activity B of the user predicted by the prediction unit 12 in step S23 is less than the threshold value (step S27: NO), the selection unit 15 selects a mode of an item, such as an image that alerts the user, stored in the storage unit 14 (step S28).

On the other hand, if the duration of activity B of the user predicted by the prediction unit 12 is greater than or equal to the threshold value (step S27: YES), the selection unit 15 selects a mode of an item that is to be presented in normal cases (step S29). Thereafter, the presentation unit 16 displays the mode of an item or the like selected by the selection unit 15, on the display device 109 (step S30).

In this way, by setting a threshold value that is based on the FIM value of a user, and performing threshold value processing on the state of the user in a certain period of time in the future predicted by the prediction unit 12, it is possible to perform more appropriate rehabilitation support before the user carries out rehabilitation.

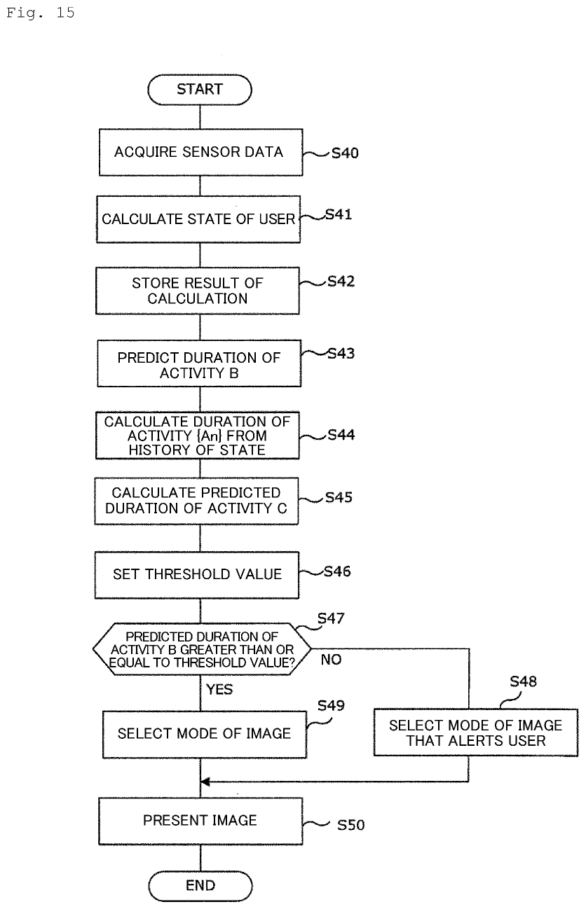

Next, as another specific example, the case of performing determination processing by using a threshold value that is based on the history of the state of the user will be described with reference to the flowchart shown in FIG. 15. For example, when a user who spends a lot of time in a lying state carries out rehabilitation for getting up or walking, it is desirable that the physical functionality of the user recovers and the duration of activity increases as a result of rehabilitation. Therefore, the determination unit 13A can set a threshold value with which the duration of activity of the user exceeds that in the past, based on the history of the state of the user. For example, a threshold value that is based on the duration of activity of the user on the previous day is set, and if a duration of activity that is lower than the duration of activity on the previous day is predicted, a mode of an image that prompts the user to carry out rehabilitation is presented.

Hereinafter, as a premise, the storage unit 14 stores a model formula that is to be used by the prediction unit 12 to predict the state of the user. First, the following processing is performed in a state where the sensor 105 is attached to the user, for example.

The sensor data acquisition unit 10 acquires the biometric information of the user measured by the sensor 105, via the transmission/reception unit 17 (step S40). The acquired biometric information is to be accumulated in the storage unit 14.

Next, the state calculation unit 11 calculates the state of the user based on the biometric information of the user, acquired by the sensor data acquisition unit 10 (step S41). The result of the calculation by the state calculation unit 11 is to be stored in the storage unit 14 together with time information (step S42). The storage unit 14 stores, for example, the history of the lying state, the get-up state, and the walking state of the user.

Thereafter, the prediction unit 12 reads out the model formula to be used to predict the state of the user from the storage unit 14, and outputs the predicted duration of activity B on the day by using, as an input, the cumulative duration of the lying state of the user calculated by the state calculation unit 11 (step S43). The duration of activity B is to be stored in the storage unit 14.

Thereafter, the determination unit 13A calculates the duration of activity {Aₙ} of the user, from the history of the state of the user calculated by the state calculation unit 11 and stored in the storage unit 14 (step S44). Here, the duration of activity on the day before the day for which the prediction value of the duration of activity of the user is to be obtained is expressed as {A₁}, and the duration of activity n days before the day is expressed as Aₙ. The determination unit 13A calculates an average value of the duration of activity {Aₙ} of the user such that the smaller n is, the larger the weight is, to calculate a predicted duration of activity C of the user in one day (step S45).

Next, the determination unit 13A sets a threshold value based on the predicted duration of activity C (step S46). Thereafter, if the duration of activity B of the user on the day, predicted by the prediction unit 12, is less than the threshold value (the predicted duration of activity C) (step S47: NO), the selection unit 15 selects a mode of an item such as an image that alerts the user, stored in the storage unit 14 (step S48).

On the other hand, if the duration of activity B of the user predicted by the prediction unit 12 is greater than or equal to the threshold value (step S47: YES), the selection unit 15 selects a mode of an item that is to be presented in normal cases (step S49). Thereafter, the presentation unit 16 displays the mode of an item or the like selected by the selection unit 15, on the display device 109 (step S50).

Note that the threshold value to be set by the determination unit 13A based on the history of the state of the user may be set based on not only the duration of activity of the user per day in the past, but also on an average in any period of time such as a week, a month, or a year. For example, when the threshold value to be used for determination processing is set based on the duration of activity of the user per day, it is possible to obtain a duration of activity that is more suitable for the current user's body by using the duration of activity of the user in the most recent day. Therefore, it is possible to set an appropriate threshold value by calculating a weighted average.

Also, as described with reference to FIG. 13, a correction value may be added to the threshold value to be used for determination processing.

In this way, by setting a threshold value that is based on the history of the state of the user, and performing threshold value processing on the state of the user in a certain period of time in the future predicted by the prediction unit 12, it is possible to perform more appropriate rehabilitation support before the user carries out rehabilitation.

### Fourth Embodiment

Next, a fourth embodiment of the present invention will be described. Note that, in the following description, the same components as in the above-described first to third embodiments are given the same reference signs and descriptions thereof are omitted.

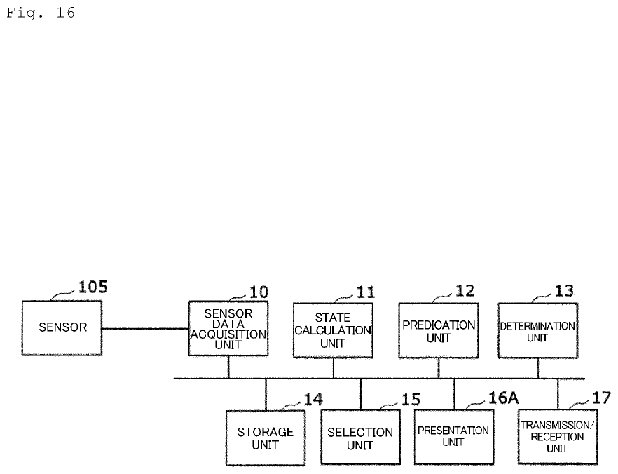

The first to third embodiments describe a case in which the selection unit 15 selects a mode of an item such as an image corresponding to the result of the determination by the determination unit 13, and the presentation unit 16 displays the selected mode of an item on the display device 109. In contrast, in the fourth embodiment, as shown in FIG. 16, the presentation unit 16A also presents a mode of an item such as an image selected by the selection unit 15 corresponding to the result of the determination to an external terminal.

For example, when a predicted value indicating that the duration of rehabilitation carried out by a user has a downward trend is output, it is possible to supports the user's rehabilitation through a third party by also presenting the trend to doctors and caregivers who manage the user's rehabilitation. Specifically, a mode of an item such as an image selected according to the result of the determination by the determination unit 13 is also presented to a communication terminal that is carried by a caregiver or the like and is connected via the communication network NW. For example, a caregiver can prompt a user who is sleeping in the day to move their body by speaking to the user, based on the presented information.

Also, when a predicted value indicating that the duration of the rehabilitation carried out by the user will be extremely long is output, the presentation unit 16A presents such information to the caregiver or the like who manages the user's rehabilitation. Thus, a third party can easily check, for example, whether or not the user's activity is excessive, or whether or not the user stays up until late at night.

Note that, as described in the first to third embodiments, a mode of an item such as an image selected according to the result of the determination by the determination unit 13 can also be presented to the user. Also, even if the result of determination is the same, the mode of an item such as an image that is to be presented to the user and the mode of an item such as an image that is to be presented to the caregiver may be different. For example, it is possible to employ a configuration with which an image and text information are presented to the user as rehabilitation support information on a display screen, and an alert sound is presented to the caregiver or the like.

Also, the presentation unit 16A may present information indicating the history of the state of the user calculated by the state calculation unit 11, in addition to the mode of an item such as an image selected according to the result of the determination by the determination unit 13.

As described above, with the rehabilitation support system according to the fourth embodiment, rehabilitation support information is presented to not only the user but also a third party that manages the user's rehabilitation. Therefore, it is possible to more reliably perform the user's rehabilitation before the user carries out the rehabilitation.

Note the above-described embodiments can be realized in combination with each other. Also, as with the first embodiment, the rehabilitation support system according to the above-described second to fourth embodiments can be realized using the sensor terminals 200a and 200b, the relay terminal 300, and the external terminal 400 shown in FIGS. 4 and 5.

The above embodiments describe cases where the relay terminal 300 in the rehabilitation support system realized using the sensor terminals 200a and 200b, the relay terminal 300, and the external terminal 400 includes the state calculation unit 11, the prediction unit 12, the determination unit 13, and the selection unit 15. However, it is possible to employ a configuration in which these functional units are included in the sensor terminals 200a and 200b or the external terminal 400.

Also, the functions of the state calculation unit 11, the prediction unit 12, the determination unit 13, and the selection unit 15 may be dispersedly realized in the sensor terminals 200a and 200b, the relay terminal 300, and the external terminal 400.

Although the embodiments of the rehabilitation support system and the rehabilitation support method according to the present invention have been described above, the present invention is not limited to the above-described embodiments, and various modifications that can be conceived of by a person skilled in the art may be applied thereto within the scope of the invention described in the claims.

### REFERENCE SIGNS LIST

10, 202 Sensor data acquisition unit
11, 303 State calculation unit
12, 304 Prediction unit
13, 305 Determination unit
14 Storage unit
15, 306 Selection unit
16, 404 Presentation unit
17 Transmission/reception unit
101 Bus
102 Processor
103 Main storage device
104 Communication interface
105, 201 Sensor
106 Auxiliary storage device
107 Clock
108 Input/output device
109 Display device
200a, 200b Sensor terminal
300 Relay terminal
400 External terminal
203, 302, 402 Data storage unit
204, 307 Transmission unit
301, 401 Reception unit
403 Presentation processing unit.

1-8. (canceled)

9. A rehabilitation support system comprising:
a sensor data acquirer configured to acquire sensor data, the sensor data comprising biometric information of a user measured by a sensor;
a state calculator configured to obtain a calculated state of the user based on the sensor data;
a predictor configured to determine a predicted state of the user based on the calculated state of the user obtained by the state calculator;
a memory configured to store support information to be presented as information that supports rehabilitation;
a selector configured to select the support information stored in the storage device based on the predicted state of the user predicted by the predictor; and
an output configured to present the support information selected by the selector.

10. The rehabilitation support system according to claim 9, wherein the state calculator is configured to calculate a cumulative duration of the calculated state of the user, and wherein the predictor predicts a cumulative duration of the predicted state of the user in a certain period in the future based on the cumulative duration of the calculated state of the user.

11. The rehabilitation support system according to claim 9, further comprising a determination device configured to determine whether or not the predicted state of the user satisfies a condition that has been set regarding execution of rehabilitation, wherein the selector is configured to select the support information corresponding to whether or not the predicted state of the user satisfies the condition.

12. The rehabilitation support system according to claim 11, wherein the determination device is configured to set a threshold value for determination, based on information that includes statistical data regarding rehabilitation for each of a plurality of users.

13. The rehabilitation support system according to claim 11, wherein the determination device is configured to set a threshold value for determination, based on a history of the calculated state of the user calculated by the state calculator.

14. The rehabilitation support system according to claim 9, wherein the output includes a display device that displays an image that expresses the support information selected by the selector.

15. The rehabilitation support system according to claim 9, wherein the predicted state of the user is a lying state, a standing state, a sitting state, or a walking state.

16. A rehabilitation support method comprising:
a first step of acquiring sensor data, the sensor data including biometric information of a user measured by a sensor;
a second step of obtaining a calculated state of the user based on the sensor data;
a third step of determining a predicted state of the user based on the calculated state of the user obtained in the second step;
a fourth step of selecting support information that is stored in a memory based on the state of the user predicted in the third step, the support information is to be presented as information that supports rehabilitation; and
a fifth step of presenting, by an output, the support information selected in the fourth step.

17. The rehabilitation support method according to claim 16, wherein the second further comprises calculating a cumulative duration of the calculated state of the user, and wherein the third step further comprises predicting a cumulative duration of the predicted state of the user in a certain period in the future based on the cumulative duration of the calculated state of the user.

18. The rehabilitation support method according to claim 16, further comprising a sixth step of determining whether or not the predicted state of the user satisfies a condition that has been set regarding execution of rehabilitation, wherein the fourth step further comprises selecting the support information corresponding to a result of the sixth step.

19. The rehabilitation support method according to claim 18, wherein the sixth step further comprises setting a threshold value for determination based on information that includes statistical data regarding rehabilitation for each of a plurality of users.

20. The rehabilitation support method according to claim 18, wherein the sixth step further comprises setting a threshold value for determination based on a history of the calculated state of the user.

21. The rehabilitation support method according to claim 16, wherein the output includes a display device that displays an image that expresses the support information selected in the fourth step.

22. The rehabilitation support method according to claim 16, wherein the predicted state of the user is a lying state, a standing state, a sitting state, or a walking state.
